(19) United States
(12) Patent Application Publication
(10) Pub. No.: US 2025/0041363 A1
(43) Pub. Date: Feb. 6, 2025

(54) COMPOSITIONS AND METHODS FOR TREATMENT OF ALCOHOL USE DISORDER AND ALCOHOL-ASSOCIATED LIVER DISEASE

(71) Applicant: UNIVERSITY OF LOUISVILLE RESEARCH FOUNDATION, INC., Louisville, KY (US)

(72) Inventor: Vatsalya VATSALYA, Louisville, KY (US)

(21) Appl. No.: 18/790,806

(22) Filed: Jul. 31, 2024

Related U.S. Application Data
(60) Provisional application No. 63/516,657, filed on Jul. 31, 2023.

(51) Int. Cl. A61K 35/747; A61K 9/51; A61K 31/404; A61K 31/4045; A61K 31/405; A61K 31/4402; A61K 31/47; A61P 25/32

(52) U.S. Cl. CPC A61K 35/747; A61K 9/5176; A61K 9/5192; A61K 31/404; A61K 31/4045; A61K 31/405; A61K 31/4402; A61K 31/47; A61P 25/32

(57) ABSTRACT

Described herein are methods of treating alcohol use disorder in a subject in need thereof. The method including administering to the subject an effective amount of a composition including: Lactobacillus rhamnosus (LGG), Lactobacillus rhamnosus (LGG)-derived exosome-like nanoparticles (LDNPs), or any combination thereof. The Lactobacillus rhamnosus (LGG)-derived exosome-like nanoparticles (LDNPs) can include tryptophan metabolites.

### CROSS-REFERENCE TO RELATED APPLICATIONS

This application claims benefit of priority of U.S. Provisional Application No. 63/516,657, filed Jul. 31, 2023, which is incorporated herein by reference in its entirety.

### STATEMENT REGARDING FEDERALLY SPONSORED RESEARCH OR DEVELOPMENT

This invention was made with government support under Grant No. K23AA029198 awarded by the National Institute of Health. The government has certain rights in the invention.

### BACKGROUND

Chronic heavy alcohol drinking is a major healthcare concern and can lead to alcohol use disorder (AUD). There are three FDA approved drugs for treating AUD: disulfiram, naltrexone and acamprosate. The FDA approved pharmacotherapies for AUD have shown limited effectiveness. Part of this problem is that the mechanisms involved in developing heavy drinking/addiction are not clear. Due to limited efficacy and adverse effects, these drugs are not widely utilized by clinicians. New/repurposed pharmacotherapeutic agents have shown low efficacy or applicability; thus, there is a need to identify effective and safe treatments for AUD.

Thus, there is an urgent need to understand the mechanisms/pathways involved in development and progression of AUD. Alterations in the gut microbiome result in a pro-inflammatory response and endotoxemia, altering the gut-brain axis, and are observed in AUD. Recent publications show that the gut dysfunction observed can alter the gut-brain axis causing the neuroinflammation involved in AUD. Recent studies have reported that treatment with probiotics can result in positive behavioral changes; however, hypothesis-driven studies of probiotic efficacy for treating AUD are lacking. Success in treating ALO is often limited due to frequent drinking relapses and lack of adherence to treatment for AUD.

Thus, a therapeutic agent that could restore normal gut function, reduce gut permeability, and resolve proinflammatory activity as a central therapeutic mechanism has the potential to improve both AUD and alcohol-associated liver disease (ALO) symptomology in a dual treatment-efficacy approach.

The compositions and methods disclosed herein address these and other needs.

### SUMMARY

Provided herein are methods of treating alcohol use disorder in a subject in need thereof. These methods can comprise administering to the subject an effective amount of a composition comprising: Lactobacillus rhamnosus (LGG), Lactobacillus rhamnosus (LGG)-derived exosome-like nanoparticles (LDNPs), or any combination thereof.

In some embodiments, the composition can comprise Lactobacillus rhamnosus (LGG). In some embodiments, the composition can comprise LDNPs. In certain embodiments, the composition can comprise LGG and LDNPs.

In some embodiments, the Lactobacillus rhamnosus (LGG)-derived exosome-like nanoparticles (LDNPs) can comprise tryptophan metabolites. In some examples the tryptophan metabolites comprise tryptophan, indole-3-aldehyde, indole-3-acetaldehyde, tryptamine, indole-3-acetic acid, xanthurenic acid, 5-hydroxy indole acetic acid, indole-3-acrylic acid, kynurenic acid, indole, indole-3-lactic acid, kynurenine, 5-hydroxytryptophan, melatonin, quinolinic acid, picolinic acid, indole-3-acetamide, or any combination thereof. In certain embodiments, the tryptophan metabolites can comprise tryptophan, indole-3-aldehyde, indole-3-acetaldehyde, tryptamine, or a combination thereof.

In some embodiments, the composition can further comprise a pharmaceutically acceptable carrier.

Also provided method of treating alcohol-associated liver disease (ALD) in a subject in need thereof. These methods can comprise administering to the subject an effective amount of a composition comprising: Lactobacillus rhamnosus (LGG), Lactobacillus rhamnosus (LGG)-derived exosome-like nanoparticles (LDNPs), or any combination thereof.

In some embodiments, the composition can comprise Lactobacillus rhamnosus (LGG). In some embodiments, the composition can comprise LDNPs. In certain embodiments, the composition can comprise LGG and LDNPs.

In some embodiments, the Lactobacillus rhamnosus (LGG)-derived exosome-like nanoparticles (LDNPs) can comprise tryptophan metabolites. In some examples the tryptophan metabolites comprise tryptophan, indole-3-aldehyde, indole-3-acetaldehyde, tryptamine, indole-3-acetic acid, xanthurenic acid, 5-hydroxy indole acetic acid, indole-3-acrylic acid, kynurenic acid, indole, indole-3-lactic acid, kynurenine, 5-hydroxytryptophan, melatonin, quinolinic acid, picolinic acid, indole-3-acetamide, or any combination thereof. In certain embodiments, the tryptophan metabolites can comprise tryptophan, indole-3-aldehyde, indole-3-acetaldehyde, tryptamine, or a combination thereof.

In some embodiments, the composition can further comprise a pharmaceutically acceptable carrier.

Also provided herein are methods of reducing alcohol craving in a subject in need thereof. These methods can comprise administering to the subject an effective amount of a composition comprising: Lactobacillus rhamnosus (LGG), Lactobacillus rhamnosus (LGG)-derived exosome-like nanoparticles (LDNPs), or any combination thereof.

In some embodiments, the composition can comprise Lactobacillus rhamnosus (LGG). In some embodiments, the composition can comprise LDNPs. In certain embodiments, the composition can comprise LGG and LDNPs.

In some embodiments, the Lactobacillus rhamnosus (LGG)-derived exosome-like nanoparticles (LDNPs) can comprise tryptophan metabolites. In some examples the tryptophan metabolites comprise tryptophan, indole-3-aldehyde, indole-3-acetaldehyde, tryptamine, indole-3-acetic acid, xanthurenic acid, 5-hydroxy indole acetic acid, indole-3-acrylic acid, kynurenic acid, indole, indole-3-lactic acid, kynurenine, 5-hydroxytryptophan, melatonin, quinolinic acid, picolinic acid, indole-3-acetamide, or any combination thereof. In certain embodiments, the tryptophan metabolites can comprise tryptophan, indole-3-aldehyde, indole-3-acetaldehyde, tryptamine, or a combination thereof.

In some embodiments, the composition can further comprise a pharmaceutically acceptable carrier.

Also provided herein are methods of inhibiting kynurenine synthesis in a subject in need thereof. These methods can comprise administering to the subject an effective amount of a composition comprising: Lactobacillus rhamnosus (LGG), Lactobacillus rhamnosus (LGG)-derived exosome-like nanoparticles (LDNPs), or any combination thereof.

In some embodiments, the composition can comprise Lactobacillus rhamnosus (LGG). In some embodiments, the composition can comprise LDNPs. In certain embodiments, the composition can comprise LGG and LDNPs.

In some embodiments, the Lactobacillus rhamnosus (LGG)-derived exosome-like nanoparticles (LDNPs) can comprise tryptophan metabolites. In some examples the tryptophan metabolites comprise tryptophan, indole-3-aldehyde, indole-3-acetaldehyde, tryptamine, indole-3-acetic acid, xanthurenic acid, 5-hydroxy indole acetic acid, indole-3-acrylic acid, kynurenic acid, indole, indole-3-lactic acid, kynurenine, 5-hydroxytryptophan, melatonin, quinolinic acid, picolinic acid, indole-3-acetamide, or any combination thereof. In certain embodiments, the tryptophan metabolites can comprise tryptophan, indole-3-aldehyde, indole-3-acetaldehyde, tryptamine, or a combination thereof.

In some embodiments, the composition can further comprise a pharmaceutically acceptable carrier.

Also provided herein are compositions that comprise Lactobacillus rhamnosus (LGG)-derived exosome-like nanoparticles (LDNPs), or Lactobacillus rhamnosus (LGG) and Lactobacillus rhamnosus (LGG)-derived exosome-like nanoparticles (LDNPs).

In some embodiments, the composition can comprise LDNPs. In certain embodiments, the composition can comprise LGG and LDNPs.

In some embodiments, the Lactobacillus rhamnosus (LGG)-derived exosome-like nanoparticles (LDNPs) can comprise tryptophan metabolites. In some examples the tryptophan metabolites comprise tryptophan, indole-3-aldehyde, indole-3-acetaldehyde, tryptamine, indole-3-acetic acid, xanthurenic acid, 5-hydroxy indole acetic acid, indole-3-acrylic acid, kynurenic acid, indole, indole-3-lactic acid, kynurenine, 5-hydroxytryptophan, melatonin, quinolinic acid, picolinic acid, indole-3-acetamide, or any combination thereof. In certain embodiments, the tryptophan metabolites can comprise tryptophan, indole-3-aldehyde, indole-3-acetaldehyde, tryptamine, or a combination thereof.

In some embodiments, the composition can further comprise a pharmaceutically acceptable carrier.

Also provided herein are methods of preparing Lactobacillus rhamnosus (LGG)-derived exosome-like nanoparticles (LDNPs). These methods can comprise culturing Lactobacillus rhamnosus; incubating Lactobacillus rhamnosus with ethanol for a period of time; and isolating LDNP.

In some embodiments, the ethanol can be present at a concentration of from 0.5% to 5% by volume, such as from 0.5% to 2.5%, from 0.5% to 2%, from 0.5% to 1%, from 1% to 2%, from 1% to 2.5%, or from 2% to 2.5%.

In some embodiments, the isolating step can comprise centrifugating at varying speeds and/or for varying times, filtering, and ultracentrifugation.

In certain embodiments, the isolating step can comprise collecting a supernatant comprising LGG and a pellet comprising LDNP after ultracentrifugation.

In certain embodiments, the isolating step can comprise washing the pellet and lyophilizing.

In some embodiments, the method can further comprise identifying the compounds in the LDNP. In certain embodiments, the identifying step can comprise a metabolic and/or proteomic analysis of the LDNP.

In some embodiments, incubating can take place for a period of time of from 24 hours to 36 hours.

### DESCRIPTION OF DRAWINGS

FIG. 1 is a graph of activity of pro-inflammatory cytokines increase downstream metabolite production of tryptophan.

FIG. 2A-2C show results of lowering of liver injury severity and progression in LGG treated mAH patients. FIG. 2A showed the 1 month change in MELD. 1-month MELD significantly dropped in the LGG arm compared to the placebo arm. FIG. 2B shows the AST:ALT ratio improvement. Drop in the AST:ALT ratio is LGG arm was significant compared to the placebo arm. FIG. 2C shows 1 month change in ABIC.

Like reference symbols in the various drawings indicate like elements.

### DETAILED DESCRIPTION

A number of embodiments of the disclosure have been described. Nevertheless, it will be understood that various modifications may be made without departing from the spirit and scope of the invention. Accordingly, other embodiments are within the scope of the following claims.

### Definitions

To facilitate understanding of the disclosure set forth herein, a number of terms are defined below. Unless defined otherwise, all technical and scientific terms used herein generally have the same meaning as commonly understood by one of ordinary skill in the art to which this disclosure belongs. Publications cited herein and the materials for which they are cited are specifically incorporated by reference.

### General Definitions

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. A range may be construed to include the start and the end of the range. For example, a range of 10% to 20% (i.e., range of 10%-20%) can includes 10% and also includes 20%, and includes percentages in between 10% and 20%, unless explicitly stated otherwise herein.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g., greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage. In particular, the term “treatment” includes the alleviation, in part or in whole, of the symptoms of coronavirus infection (e.g., sore throat, blocked and/or runny nose, cough and/or elevated temperature associated with a common cold). Such treatment may include eradication, or slowing of population growth, of a microbial agent associated with inflammation.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount”. However, an appropriate “effective’ amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n-COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician. Administration of the therapeutic agents can be carried out at dosages and for periods of time effective for treatment of a subject. In some embodiments, the subject is a human.

Reference will now be made in detail to specific aspects of the disclosed materials, compounds, compositions, articles, and methods, examples of which are illustrated in the accompanying Examples and Figures.

### Compositions

Described herein are compositions including: Lactobacillus rhamnosus (LGG), Lactobacillus rhamnosus (LGG)-derived exosome-like nanoparticles (LDNPs), or any combination thereof.

In some embodiments, the Lactobacillus rhamnosus (LGG)-derived exosome-like nanoparticles (LDNPs) can include tryptophan metabolites. Tryptophan metabolites can include, but are not limited to, tryptophan, indole-3-aldehyde, indole-3-acetaldehyde, tryptamine, indole-3-acetic acid, xanthurenic acid, 5-hydroxy indole acetic acid, indole-3-acrylic acid, kynurenic acid, indole, indole-3-lactic acid, kynurenine, 5-hydroxytryptophan, melatonin, quinolinic acid, picolinic acid, indole-3-acetamide, or any combination thereof. In certain embodiments, the tryptophan metabolites can comprise tryptophan, indole-3-aldehyde, indole-3-acetaldehyde, tryptamine, or a combination thereof.

In some embodiments, the compositions can include LGG. In some embodiments, the composition can include LDNPs. In some embodiments, the compositions can include LGG and LDNPs.

In some embodiments, the compositions can further include a pharmaceutically acceptable carrier.

### Methods of Use

Described herein are methods of treating alcohol use disorder and/or alcohol-associated liver disease in a subject in need thereof. In some embodiments, described herein are methods of treating alcohol use disorder in a subject in need thereof. In some embodiments, described herein are methods of treating alcohol-associated liver disease in a subject in need thereof.

Described herein are also methods of reducing alcohol craving in a subject in need thereof. Also described herein are methods of inhibiting kynurenine synthesis in a subject in need thereof.

In some embodiments, the methods can include administering to the subject an effective amount of a composition described herein.

### Methods of Making

Described herein are methods of preparing Lactobacillus rhamnosus (LGG)-derived exosome-like nanoparticles (LDNPs). The method can include: culturing Lactobacillus rhamnosus; incubating Lactobacillus rhamnosus with ethanol for a period of time; and isolating LDNPs.

In some embodiments, the ethanol concentration can be present at a concentration of from 0.5% to 5% by volume, such as from 0.5% to 2.5%, from 0.5% to 2%, from 0.5% to 1%, from 1% to 2%, from 1% to 2.5%, or from 2% to 2.5%.

In some embodiments, incubating can take place for a period of time of from 24 to 36 hours at 37° C.

In some embodiments, the isolating step comprises centrifugating at varying speeds and times, such as 2,500 G at 4° C. for ¼ hours, followed by 5,000 for ¼ hours, and 10,000 G for ½ hours to eliminate debris. In some embodiments, the isolating step can be followed by collecting, filtering, and ultracentrifugation, such as ultracentrifugation at 100,000 G for 1 hour.

In some embodiments, the isolating step can further include collecting a supernatant including LGG and a pellet including LDNP after ultracentrifugation. In some embodiments, the isolating step can include washing the pellet and lyophilizing.

In some embodiments, the method can further include identifying the compounds in the LDNP, for example, using a metabolic and/or proteomic analysis of the LDNP.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically related may be substituted for the agents described herein while the same or similar results would be achieved. All such similar substitutes and modifications apparent to those skilled in the art are deemed to be within the spirit, scope and concept of the disclosure as defined by the appended claims.

By way of non-limiting illustration, examples of certain embodiments of the present disclosure are given below.

### EXAMPLES

### Example 1: Kynurenic Acid as a Therapeutic-Mechanistic Target for Alcohol Use Disorder: Reward Domain of Alcohol Addiction

Many cells of the immune system regulate Tryptophan metabolites and participate in various immune-mediated diseases and disorders. Kynurenic acid (KYNA) is a product of the kynurenine pathway of tryptophan metabolism and its influences impart several neurophysiological and neuropathological processes. The intimate connection of KYNA to the immune system, inflammation, and cancer has been evolving. Especially KYNA's anti-inflammatory and immunosuppressive functions are impressive, and its studies will unravel more interesting effects on biological functions. The biological functions of KYNA on its immunomodulatory characteristics by signaling via G-protein-coupled receptor 35 (GPR35)- and aryl hydrocarbon receptor-mediated pathways are worth studying. More interestingly the role of KYNA-GPR35 interaction and microbiota associated KYNA metabolism in AUD to maintain gut homeostasis is sharply evolving. The degradation of tryptophan (TRP) in the Kynurenine pathway (KP) exerts a crucial role in the regulation of the immune function, with respect to a counter-regulatory mechanism especially in inflammation. Three rate-limiting enzymes of KP, tryptophan 2,3-dioxygenase (TDO) and indolamine 2,3-dioxygenase (IDO) 1 and 2, have been described so far TDO is positively regulated by TRP in order to maintain the homeostasis of TRP. The expression and activity of TDO is sharply regulated by hormones such as cortisol, insulin, glucagon, or epinephrine. ID01 and 2 are also upregulated by Inflammatory stimuli such as interferon-γ (IFN-γ). The significance of KP activation solely depends on the production of active metabolites such as kynurenine (KYN), kynurenic acid (KYNA), quinolinic acid (QUIN), or anthranilic acid mediating some immune- and neuromodulative functions. Within the central nervous system, it has been well documented that metabolites such as KYNA and QUIN control some neurological functions. Thus, KYNA acts as an antagonist affecting certain ionotropic glutamate receptors including NMDA, AMPA, and kainate receptors as well as the α7 nicotinic acetylcholine receptor (α7nAChR) considering it as a neuroprotective metabolite. However, the Inhibition of α7nAChR by KYNA is debated because some studies have shown this mechanism could not repeated the original results. Dysregulation of KP, resulting in alterations of the balance between KYNA and QUIN, has been described in many neurological disorders.

However, alterations of KYNA are also described in several inflammatory-related states, such as sepsis or inflammatory bowel disease (IBD) and are discussed as a potential marker in cancer. It is generally accepted that KYNA mediates immunosuppressive effects, particularly targeting the G-protein-coupled receptor 35 (GPR35)- or aryl hydrocarbon receptor (AhR)-associated signaling pathways.

G-protein-coupled receptor 35 is expressed in various subpopulations of immune cells, including peripheral monocytes, mast cells, basophils, eosinophils, and INKT cells. A high level of GPR35 expression was detected throughout the digestive tract, as well as in lung, skeletal muscle, uterus, and dorsal root ganglion. Moderate expression was found in heart, liver, bladder, spinal cord, whole brain, and cerebrum.

Recently, it was found that GPR35 is a high-affinity receptor for the mucosal chemokine CXCL17. Nevertheless, KYNA was the first reported agonist ligand for GPR35. This was identified by high-throughput screening using changes of intracellular calcium (Ca2+) in the Chinese hamster ovary cell line, CHO, co-expressing GPR35 and a G-protein mixture as a readout. Further in-depth studies revealed that KYNA-GPR35 Interaction Inhibited N-type Ca2+ channels in sympathetic neurons and reduced the plateau phase of ATP-induced calcium transients in astrocytes. The later study also demonstrated that KYNA-mediated GPR35 activation decreased forskolin-induced cAMP elevation. Furthermore, the recruitment of β-arrestin 2 mediated GPR35 internalization upon KYNA activation, which led to receptor desensitization.

Kynurenic acid may also have an inhibitory effect on the phosphoinositide 3-kinase (P13K)/protein kinase B (Akt) and mitogen-activated protein kinase (MAPK) pathways. Walczak et al. demonstrated that KYNA decreased phosphorylation oi extracellular signal-regulated kinases (ERK) 1/2, p38 MAPK, and Akt in colon epithelial cells. They also found indications that KYNA Induced accumulation of β-catenin. MAPK, P13K/Akt and β-catenin pathways are well-known targets of GPR signaling. Therefore, it is possible that the observed inhibition of ERK and p38, as well as the induction of β-catenin accumulation after KYNA treatment, are a consequence of GRP35 activation. Interestingly, all of these described effects of KYNA-GPR35 signaling might lead to the suppression or limitation of inflammation.

Increased intracellular calcium is associated with inflammatory signal secretion and triggers the activation of NF-KB, which is an essential transcription factor in inflammation. The CAMP pathway is known to regulate innate and adaptive immune cell activities [e.g., T-cell functions]. In this respect, there is strong evidence that KYNA-GPR35-mediated inhibition of adenylate cyclase is causal for the downregulation of the IL23/IL17 immune axis observed after KYNA treatment. Furthermore, the P13K/Akt pathway and MAPK's play crucial roles in generating an inflammatory response Conversely, the β-catenin signaling pathway is known to inhibit inflammation through limiting NF-KB activation by stabilizing the NF-KB inhibitory IkB-factors.

KYNA is intimately involved by microbiota in gut homeostasis. High GPR35 expression in the gastrointestinal tract Indicates that this receptor, and its ligand KYNA. could have a function in gut homeostasis. The potential significance of KYNA for gut health emerges from its association with various bowel diseases and colon cancer. as well as the potential anti-inflammatory effects of KYNA treatment in dogs with experimental colon obstruction. Studies In rats and pigs have shown a high concentration of KYNA in the intestinal lumen. The intestinal KYNA concentration increased from the proximal to the distal part of the gut, reaching ˜16 μM in the distal ileum of the rat and ˜1.6 μMin the colon of the pig. Preclinical studies with rats suggest that appropriate amounts of KYNA in the gut originated from the intestinal microflora, due to the relatively low concentrations in the wall of the ileum (˜0.2-0 3 μM) and the food (˜0.6 μM). Certain foods and herbs may contain relatively high amounts of KYNA like broccoli (˜2 μM), honey (˜1 μM), basil (˜74 μM), and thyme (˜9 μM). The intestinal commensal Escherichia coli can produce and liberate KYNA through aspartate aminotransferase (AspAT) and it is readily absorbed from the gut into the bloodstream. Studies with rats using probiotic Bifidobacteria infantis in the gut have significantly higher KYNA levels in the blood then un-colonized control animals. Blood from B. infantis-colonized animals exhibits a lower TNF induction after ex vivo challenge with LPS, which is a typical indication of an endotoxin tolerance and are also clear indications that KYNA selectively regulates the growth, and thereby the composition, of the intestinal microbiota. So far the microbial-mediated KYNA catabolism from Pseudomonas and Aerococcus, studies might be relevant. Feeding with very high amounts of KYNA might have a toxic/stress-inducing effect in rainbow trout. Further studies are essential to evaluate if a supplementation of KYNA is beneficial or detrimental to human health. The proven fact and relevance of KYNA for various diseases, it is often mooted as both a target and agent for therapeutic interventions. But the interference of KYNA with diverse immune-related signaling pathways requires in-depth analysis to avert unexpected adverse consequences. We used this background to explore its therapeutic involvement in Alcohol use Disorder (AUD).

A pathological pathway model of gut-brain axis has been pioneered by our group simultaneously.

Preliminary results of our pilot study showed a reduction of heavy drinking markers in AUD patients treated for 6-months with the probiotic, Lactobacillus rhamnosus GG (LGG).

Importantly, heavy drinkers frequently suffer from alcohol-associated liver disease (ALO). Heavy and chronic drinking is involved in both early-stage and advanced forms of ALD. Success of medical management of ALD is limited due to frequent drinking relapses, and lack of treatment adherence. Moreover, there is no FDA-approved therapy for any stage of ALO. Increasing evidence suggests that gut-microbiome modulating probiotics or metabolites derived from probiotics can ameliorate alcohol-associated liver injury in animal models, and humans by restoring gut microflora and fecal metabolites. The beneficial effects of probiotic LGG against AUD can be mediated by LDNPs.

LGG and its candidate metabolites could be used as a therapy to alleviate heavy and chronic drinking as observed in AUD. A therapy that would treat both AUD and ALD is highly desirable. Tryptophan catabolic metabolites exert their functional effects on alcohol-induced bacterial translocation, this mechanism could be one of the targets for therapeutic assessment.

Tryptophan pathway associated LDNP: targeting neuronal activity for heavy drinking. L-tryptophan is an essential amino acid that helps the body make proteins and certain brain-signaling chemicals. Depletion of Tryptophan could induce loss of control, impulsivity and cause problematic patterns of drinking (neuroadaptive changes due to neuroinflammation). Targets for inhibiting kynurenine synthesis using specific LDNPs can include Indoleamine 2,3-Dioxygenase (IDO) antagonist (Inhibitors), and supplementation of LGG synthesized Kynurenic acid (KYNA) as LDNP product (to target allosteric modulation of the Tryptophan downstream pathway).

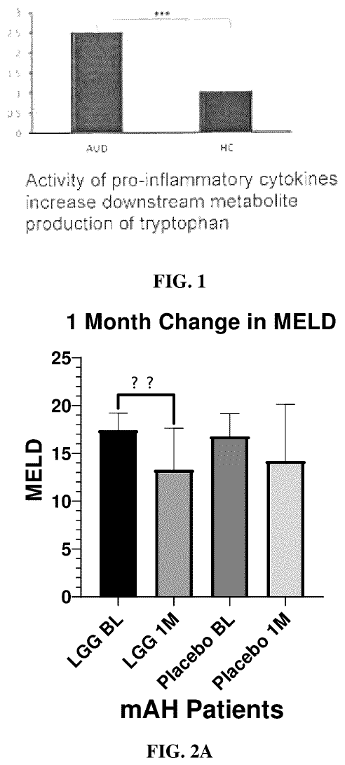

Many pro-inflammatory cytokines and LPS activity that have been discussed earlier are shown to get attenuated in the experimental results shown in FIG. 1.

The beneficial effects of probiotic LGG against AUD can be mediated by LDNPs. We have the ability to produce the metabolite that can be used as therapeutic intervention. Isolation of LDNPs: LDNPs can be isolated using ultracentrifugation and they can be characterized by metabolomic and proteomic analysis and microscopic imaging. Metabolomic studies characterized many metabolites in LDNPs cargo. Several indole derivatives, which are tryptophan metabolites.

Extraction process: LGG was purchased from American Type Culture Collection (ATCC 53103, Rockville, MD) and cultured in autoclaved deMan, Rogosa and Sharpe (MRS) broth at 37° C. for 40 hours. The culture density was measured with a spectrophotometer at OD600. The culture suspension (2×109 CFU/ml) was centrifuged at 2,000 g for 10 minutes, at 5,000 g for 20 minutes, and then at 10,000 g for 30 minutes to eliminate debris including dead cells and other waste materials. The obtained supernatant was filtered and ultracentrifuged at 150,000 g for 70 minutes (Optima L-100XP Ultra Centrifuge; Bechman Coulter, Atlanta, GA). After ultracentrifugation, the supernatants were collected and stored (nanoparticle-depleted LGGs, or LGGs [np-d]), and the pellet containing LD Ps was washed in phosphate-buffered saline (PBS), ultracentrifuged, resuspended in PBS, and stored at −80° C. for later use.

Extraction Method for Developing Targeted Drug (LGG Derived Nanoparticles, LDNP) from Lactobacillus GG Sensitized with Alcohol

LGG of 5×109 CFU/mL (each unit dose) was loaded in a 100 ml test tube batch with 50 ml MRS (De Mann-Rogosa-Sharpe) media solution. 5 sets of test-tubes with the 5 ml solution of 0.5%, 1%, 2% 2.5%, 5% EtOH were added in each. The test-tubes were kept at 37° C. for 36 hrs. incubation. The batch culture density was observed as healthy with a spectrometer at the OD600 of 1.0-2.0. The TT assembly is centrifuged at 2,500 G at 4° C. for ¼ Hr. followed by 5,000 for ¼ Hr. and 10,000 G for ½ Hr to eliminate debris. The supernatant was then collected, filtered, and ultracentrifuged at 100,000 G for 1 Hr. Post-ultracentrifugation, the supernatant is collected as LGG without any LDNP. This was used to repeat the incubation step to estimate the culture estimates without the alcohol exposure for viability of the culture post-exposure. (If colony survived/thrives with an alcohol environment). The pellet containing LDNP is collected separately and washed with 0.9% sodium chloride solution and lyophilized.

Results: The test with 2.5% alcohol exposure yields maximum targeted synthesis of the substance. LGG thrives in the combination media of MRS and alcohol solution at all concentrations. Tables 1-3 show metabolic analysis and identification of the LDNPs (A is the group with paper labeling, P is the group without paper labeling).

TABLE 1

| | | Compound name | | | | | |
|---|---|---|---|---|---|---|---|
| | | | | | | indole-3- | |
| | | | indole-3- | indole-3- | | acetic | xanthurenic |
| | | Tryptophan | aldehyde | acetaldehyde | tryptamine | acid | acid |
| Retention time | | 4.45 | 7.05 | 6.87 | 5.36 | 7.1 | 6.89 |
| Peak area | A0 | 587256.625 | 50132.605 | 665.872 | 790.314 | 1325.187 | 198.531 |
| | A1 | 1019284.06 | 36882.773 | 538.155 | 585.146 | 1038.95 | 156.436 |
| | A2.5 | 955488.813 | 31537.615 | 28.573 | 594.034 | 1092.897 | 84.832 |
| | A5 | 909211.875 | 27119.25 | 264.544 | 588.21 | 1110.129 | 56.815 |
| | A0.5 | 999164.875 | 43288.707 | 408.383 | 605.322 | 963.436 | 135.141 |
| | P0 | 991062.25 | 57164.438 | 2103.574 | 0 | 1060.411 | 695.33 |
| | P1 | 910304.25 | 44234.488 | 2109.003 | 600.396 | 844.82 | 871.927 |
| | P2.5 | 949636.438 | 48303.129 | 1488.221 | 655.165 | 1032.607 | 865.58 |
| | P5 | 940369.875 | 33883.406 | 1371.656 | 771.811 | 1107.262 | 585.756 |
| | P0.5 | 908323.5 | 37094.754 | 1930.826 | 648.441 | 850.68 | 651.823 |

TABLE 2

| | | 5-hydroxy | | | | |
|---|---|---|---|---|---|---|
| | | indole | indole-3- | | | indole-3- |
| | | acetic | acrylic | kynurenic | | lactic |
| Compound name | | acid | acid | acid | indole | acid |
| Retention time | | 7.09 | 7.05 | 5.2 | 7.06 | 6.87 |
| Peak | A0 | 1491.01 | 207.482 | 2468.564 | 16639.668 | 3243.075 |
| area | A1 | 1182.383 | 344.417 | 1993.889 | 15292.579 | 2235.601 |
| | A2.5 | 959.441 | 371.559 | 1810.681 | 13988.92 | 2196.772 |
| | A5 | 986.598 | 289.461 | 1642.771 | 12480.461 | 952.237 |
| | A0.5 | 1351.417 | 255.087 | 2224.903 | 16343.835 | 2493.641 |
| | P0 | 1337.238 | 362.849 | 7154.686 | 22316.531 | 13351.56 |
| | P1 | 1592.308 | 420.801 | 5482.145 | 15943.32 | 13882.37 |
| | P2.5 | 1541.04 | 303.37 | 4799.935 | 17007.996 | 11485.85 |
| | P5 | 1412.526 | 437.482 | 3482.25 | 17909.793 | 8946.11 |
| | P0.5 | 1235.547 | 373.354 | 5122.642 | 15573.095 | 9705.234 |

TABLE 3

| | | Compound name | | | | | |
|---|---|---|---|---|---|---|---|
| | | | 5-hydroxy- | | quinolinic | picolinic | indole-3- |
| | | kynurenine | tryptophan | melatonin | acid | acid | acetamide |
| Retention time | | 3.35 | 3.09 | 6.67 | 2.35 | 2.96 | 6.68 |
| Peak area | A0 | 1191.92 | 410.704 | 0 | 460.008 | 141.161 | 68.269 |
| | A1 | 1021.998 | 447.075 | 86.062 | 455.912 | 205.046 | 68.102 |
| | A2.5 | 1040.852 | 383.381 | 121.384 | 639.147 | 169.614 | 31.894 |
| | A5 | 1044.294 | 355.614 | 160.488 | 666.88 | 240.072 | 48.319 |
| | A0.5 | 970.269 | 391.172 | 81.955 | 461.116 | 217.238 | 70.362 |
| | P0 | 8210.803 | 815.292 | 62.127 | 987.341 | 81.645 | 32.72 |
| | P1 | 6975.019 | 518.412 | 1043.204 | 556.054 | 216.752 | 43.407 |
| | P2.5 | 7130.862 | 556.414 | 2826.825 | 458.095 | 114.677 | 57.205 |
| | P5 | 6338.477 | 422.922 | 3980.215 | 701.822 | 210.128 | 63.361 |
| | P0.5 | 7226.674 | 745.6 | 527.414 | 716.005 | 221.047 | 30.819 |

### Example 2: The Beneficial Effects of Lactobacillus GG Therapy on the Gut-Liver Axis and Drinking Behavior in Experimental Mice and Patients with Moderate Alcohol-Associated Hepatitis

High morbidity and mortality as well as a huge burden on the healthcare resources in patients with alcohol use disorder (AUD) and alcohol-associated liver disease (ALD). Frequent relapses in drinking among patients with AUD and ALD exacerbate ALD and worsen prognosis. An intervention with the potential to treat both AUD and ALD is a major unmet need. Gut dysfunction is considered a central mechanism of pro-inflammatory activity observed in the neuroinflammation in AUD, and of the liver inflammation/injury in ALD.

Gut barrier dysfunction and dysbiosis have been postulated to play an etiologic role in ALD for >half century. Gut barrier dysfunction and dysbiosis have been postulated to play a role in neuroinflammation in patients with AUD (Leclercq, 2014). Gut barrier dysfunction and dysbiosis have been postulated to play a role in alcohol craving (Wang, 2020). Human studies are lacking concerning whether treatment of gut barrier dysfunction improves neuroinflammation and craving in patients with AUD. The pathophysiological connectivity of craving and withdrawal illustrated by the gut-brain axis in AUD is illustrated by Vatsalya, McClain et al., 2022.

### Experimental

Conduct a pilot double-blind treatment trial identifying the dual therapeutic role of LGG in patients with comorbid AUD and moderate AH. Evaluate the therapeutic effects of LGG on lowering alcohol consumption over 6 months of treatment compared to placebo. Evaluate the therapeutic outcomes of LGG treatment on liver injury, progression and prognosis compared to placebo. Validate the central role of gut dysfunction in the altered gut-liver and gut-brain response in patients with AUD and moderate AH.

We analyzed data from 46 participants in a large NIAAA-funded clinical trial performed at 4 medical centers (DASH Consortium). This investigation was a double-blind placebo-controlled study of patients with moderately severe acute alcohol associated hepatitis (mAH, MELD <21) who had pre-existing/ongoing heavy alcohol drinking, with clinical assessments at multiple time points. Data on demographics, drinking, and liver injury were collected and assessed at baseline (BL), 1-month (1-m), 3-months (3-m), and 6-months (6-m). Alcohol consumption was measured by baseline AUDIT, monthly alcohol use questionnaire; and liver injury was assessed by standard liver injury markers. All patients received standard medical management for liver disease and counseling for AUD in the standard of care protocol.

Table 4 shows demographic, drinking and clinical presentation for placebo and active LGG.

TABLE 4
Placebo (N = 22)
Baseline Demographics, Drinking; and Candidate Liver Severity and Injury Measures

| | AUDIT | | BMI | | | Maddrey | |
|---|---|---|---|---|---|---|---|
| Sex | (Unit | Age | (Unit | | LTDH | (Unit | Platelets |
| (F %) | Score) | (years) | Score) | Race | (years) | Score) | (10\hat{ }9/L) |
| 11 | 25.05 ± | 46 ± 9.8 | 26 ± 5.5 | 19 W, | 23.0 ± | 18.18 ± | 10.91 ± |
| (50%) | 9.68 | | | 1 A, | 13.58 | 10.3 | 1.25 |
| | | | | 2 AAs | | | |

Active LGG (N = 24)
Baseline Demographics, Drinking; and Candidate Liver Severity and Injury Measures

| | AUDIT | | BMI | | | Maddrey | |
|---|---|---|---|---|---|---|---|
| Sex | (Unit | Age | (Unit | | LTDH | (Unit | Platelets |
| (F %) | Score) | (years) | Score) | Race | (years) | Score) | (10\hat{ }9/L) |
| 9 | 23.18 ± | 44 ± 12 | 30 ± 7.6 | 23 W, | 23.18 ± | 21.80 ± | 10.49 ± |
| (37.5%) | 8.37 | | | 1 AA | 8.38 | 13.13 | 1.38 |

Drinking, Liver-Injury and Severity Measures Over the Treatment Course of 6-months

| Timeline | Baseline | 1 month | 3 month | 6 month | Baseline | 1 month | 3 month | 6 month |
|---|---|---|---|---|---|---|---|---|
| #Drinks | 42.37 ± 23.44 | 1.14 ± 2.72 | 5.59 ± 15.7 | 5.86 ± 8.3 | 34.71 ± 21.99 | 1.81 ± 3.77 | 2.83 ± 7.71 | 0.31 ± 1.01 |
| (Weekly) | | (n = 17) | (n = 11) | (n = 7) | | (n = 23) | (n = 18) | (n = 16) |
| MELD (Unit | 16.77 ± 2.39 | 14.19 ± 5.95 | 11.60 ± 4.9 | 9.14 ± 2.8 | 17.42 ± 1.79 | 13.29 ± 4.34 | 12.06 ± 7.73 | 12.40 ± 8.4 |
| score) | | (n = 16) | (n = 10) | (n = 7) | | (n = 17) | (n = 17) | (n = 15) |
| ABIC (Unit | 6.51 ± 1.05 | 6.16 ± 1.2 | 5.90 ± 1.0 | 5.87 ± 0.7 | 6.3 ± 1.26 | 5.84 ± 1.2 | 5.98 ± 1.5 | 6.08 ± 1.46 |
| Score) | | (n = 17) | (n = 11) | (n = 7) | | (n = 17) | (n = 17) | (n = 16) |
| Total | 9.2 ± 7.5 | 3.96 ± 4.4 | 1.85 ± 1.3 | 1.53 ± 0.7 | 7.2 ± 4.5 | 2.54 ± 1.4 | 3.82 ± 8.3 | 2.8 ± 3.84 |
| Bilirubin | | (n = 17) | (n = 11) | (n = 7) | | (n = 18) | (n = 17) | (n = 15) |
| (mg/dl) | | | | | | | | |
| Albumin | 2.8 ± 0.5 | 3.3 ± 0.6 | 3.71 ± 0.7 | 3.93 ± 0.7 | 2.7 ± 0.6 | 3.5 ± 0.8 | 3.6 ± 0.9 | 4.01 ± 0.94 |
| (g/dL) | | (n = 17) | (n = 11) | (n = 7) | | (n = 18) | (n = 17) | (n = 16) |
| AST (U/L) | 165 ± 81.8 | 93.6 ± 86.8 | 37.09 ± 15.6 | 51.86 ± 27.3 | 169.0 ± 88.4 | 71.72 ± 59.9 | 58.65 ± 51.9 | 48.75 ± 25.20 |
| | | (n = 17) | (n = 11) | (n = 7) | | (n = 18) | (n = 17) | (n = 16) |
| ALT (U/L) | 53.8 ± 33.5 | 41.8 ± 43.4 | 36.27 ± 35.4 | 32.86 ± 10.7 | 73.7 ± 46.7 | 43.6 ± 44.9 | 33.39 ± 36.27 | 38.75 ± 32.48 |
| | | (n = 17) | (n = 11) | (n = 7) | | (n = 18) | (n = 17) | (n = 16) |
| AST:ALT | 3.45 ± 1.3 | 2.54 ± 1.0 | 1.51 ± 0.7 | 1.61 ± 0.7 | 2.74 ± 1.54 | 1.99 ± 1.2 | 1.7 ± 0.82 | 1.65 ± 0.97 |
| | | (n = 17) | (n = 11) | (n = 7) | | (n = 18) | (n = 17) | (n = 16) |
| Creatinine | 0.61 ± 0.25 | .75 ± 0.4 | 0.88 ± 0.7 | 0.66 ± 0.2 | 0.64 ± 0.17 | .81 ± 0.5 | 0.90 ± 0.4 | 0.88 ± 0.35 |
| (mg/dL) | | (n = 17) | (n = 10) | (n = 7) | | (n = 18) | (n = 17) | (n = 16) |
| INR (Unit | 1.382 ± 0.24 | 1.36 ± 0.4 | 1.22 ± 0.2 | 1.13 ± 0.1 | 1.453 ± 0.35 | 1.32 ± 0.3 | 1.31 ± 0.45 | 1.41 ± 0.69 |
| Score) | | (n = 17) | (n = 10) | (n = 7) | | (n = 17) | (n = 17) | (n = 16) |

Patients treated with LGG showed a significant reduction in drinking, to the social or abstinence level of <4 drinks/week at the 6-month assessment compared to the patients treated with placebo. There was a significant drop (p=0.0128) at the 6-month assessment for average drinks/week in LGG arm compared to the placebo arm. Lowering of alcohol consumption over 6 months in both the groups for average drinking/week was significant, with higher significance (p≤0.001) in the LGG group. It is likely that counseling (part of standard of care) is a contributing factor to the reduction in alcohol intake in the placebo arm. Outcome: sixteen of the 24 LGG-treated patients and four of the 22 Placebo treated patients ultimately had a reduction of alcohol drinks/week to the level of social drinking criteria. Significant distribution analysis (Fisher's exact test, p=0.0012; Likelihood ratio=9.0 [moderate-effect]) for the LGG treated patients supports recovery to social drinking or abstinence level. Compliance and Adherence to Study: nine of 22 Placebo treated patients and eight of the 24 LGG-treated patients did not continue with the medical management through the full six months.

Probiotics are live microorganisms (bacteria or yeasts) which, when administered in adequate amounts, confer a health benefit on the host. Lactobacillus GG-bacteria, supernatant, exosome increases intestinal trefoil factor, antimicrobial peptides, intestinal AhR/IL-22, intestinal FXR/FGF 19/15, and AMPK; and decreases endotoxemia, proinflammatory cytokines, liver apoptosis, hepatic fat/inflammation, and hepatic fibrosis. Jiang, et al, 2022 (Hepatology) shows the efficacy of probiotic derived nanoparticles to treat ALD in mice alleviating liver injury.

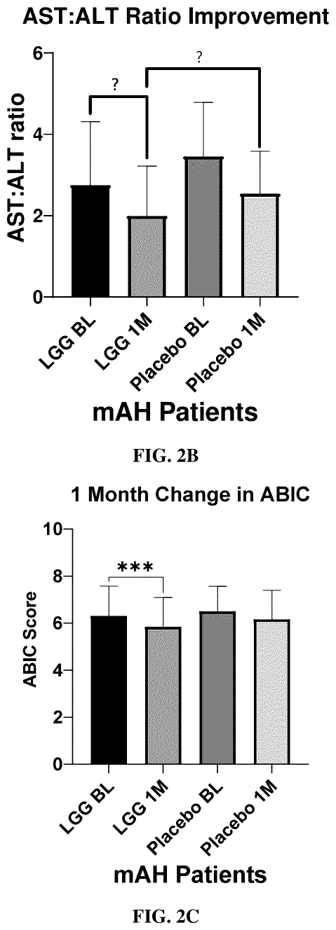

Results of lowering of liver injury severity and progression in LGG treated mAH patients are shown in FIGS. 2A-2C. FIG. 2A showed the 1 month change in MELD. 1-month MELD significantly dropped in the LGG arm compared to the placebo arm. FIG. 2B shows the AST:ALT ratio improvement. Drop in the AST:ALT ratio is LGG arm was significant compared to the placebo arm. FIG. 2C shows 1 month change in ABIC. 1-month ABIC score dropped significantly (p=0.005) in LGG arm. In summary, 6 months of LGG treatment reduced heavy drinking levels to the social drinking or abstinence levels. Counseling therapy in patients, particularly in the placebo treated patients with AUD, seems to provide some benefit in reducing drinking levels. LGG treatment showed a significant reduction of some biomarkers of liver injury progression and severity. Results warrant a larger well-designed, rigorous treatment-trial clinical study, which we are presently pursuing: K23AA029198 (VV).

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

What is claimed is:

1. A method of treating alcohol use disorder in a subject in need thereof, a method of treating alcohol-associated liver disease in a subject in need thereof, a method of reducing alcohol craving in a subject in need thereof, a method of inhibiting kynurenine synthesis in a subject in need thereof, or any combination thereof, the method comprising:
administering to the subject an effective amount of a composition comprising: Lactobacillus rhamnosus (LGG), Lactobacillus rhamnosus (LGG)-derived exosome-like nanoparticles (LDNPs), or any combination thereof,
wherein the Lactobacillus rhamnosus (LGG)-derived exosome-like nanoparticles (LDNPs) comprise tryptophan metabolites.

2. The method of claim 1, wherein the composition comprises LGG.

3. The method of claim 1, wherein the composition comprises LDNPs.

4. The method of claim 1, wherein the composition comprises LGG and LDNPs.

5. The method of claim 1, wherein the tryptophan metabolites comprise tryptophan, indole-3-aldehyde, indole-3-acetaldehyde, tryptamine, indole-3-acetic acid, xanthurenic acid, 5-hydroxy indole acetic acid, indole-3-acrylic acid, kynurenic acid, indole, indole-3-lactic acid, kynurenine, 5-hydroxytryptophan, melatonin, quinolinic acid, picolinic acid, indole-3-acetamide, or any combination thereof.

6. The method of claim 1, wherein the composition further comprises a pharmaceutically acceptable carrier.

7. A method of preparing Lactobacillus rhamnosus (LGG)-derived exosome-like nanoparticles (LDNPs), the method comprising:
culturing Lactobacillus rhamnosus;
incubating Lactobacillus rhamnosus with ethanol for a period of time; and
isolating the LDNPs.

8. The method of claim 7, wherein the ethanol concentration of from 0.5% to 5% ethanol, such as from 0.5% to 2.5%, from 0.5% to 2%, from 0.5% to 1%, from 1% to 2%, from 1% to 2.5%, or from 2% to 2.5%.

9. The method of claim 7, wherein the isolating step comprises centrifugating at varying speeds and/or for varying times, filtering, and ultracentrifugation.

10. The method of claim 7, wherein the isolating step comprises collecting a supernatant comprising LGG and a pellet comprising LDNP after ultracentrifugation.

11. The method of claim 10, wherein the isolating step comprises washing the pellet and lyophilizing.

12. The method of claim 7, further comprising identifying compounds in the LDNP.

13. The method of claim 12, wherein the identifying step comprises a metabolic and/or proteomic analysis of the LDNP.

14. The method of claim 7, wherein incubating comprising incubating the Lactobacillus rhamnosus with the ethanol for a period of time of from 24 hours to 36 hours.

15. A composition comprising:
Lactobacillus rhamnosus (LGG)-derived exosome-like nanoparticles (LDNPs), or Lactobacillus rhamnosus (LGG) and Lactobacillus rhamnosus (LGG)-derived exosome-like nanoparticles (LDNPs),
wherein the Lactobacillus rhamnosus (LGG)-derived exosome-like nanoparticles (LDNPs) comprise tryptophan metabolites.

16. The method of claim 15, wherein the composition comprises LDNPs.

17. The method of claim 15, wherein the composition comprises LGG and LDNPs.

18. The method of claim 15, wherein the tryptophan metabolites comprise tryptophan, indole-3-aldehyde, indole-3-acetaldehyde, tryptamine, indole-3-acetic acid, xanthurenic acid, 5-hydroxy indole acetic acid, indole-3-acrylic acid, kynurenic acid, indole, indole-3-lactic acid, kynurenine, 5-hydroxytryptophan, melatonin, quinolinic acid, picolinic acid, indole-3-acetamide, or any combination thereof.

19. The method of claim 15, wherein the composition further comprises a pharmaceutically acceptable carrier.
